(19) United States
(12) Patent Application Publication
(10) Pub. No.: US 2004/0086583 A1
(43) Pub. Date: May 6, 2004

(54) Anti-angiogenesis effects of morinda citrifolia

(76) Inventors: Claude Jarakae Jensen, Cedar Hills, UT (US); Afa Kehaati Palu, Orem, UT (US); David Lemus, Santiogo (CL); Marcela Fuenzalida, Santiago (CL); Danilo Mihovilovic, Nonoa Stgo (CL)

Correspondence Address: KIRTON AND MCCONKIE, 1800 EAGLE GATE TOWER, 60 EAST SOUTH TEMPLE, P O BOX 45120, SALT LAKE CITY, UT, 84145-0120, US

(21) Appl. No.: 10/286,112

(22) Filed: Nov. 1, 2002

(51) Int. Cl. A61K 35/78

(52) U.S. Cl. 424/765; 424/769; 424/777

(57) ABSTRACT

The present invention features a method of inducing an anti-angiogenesis effect within a tumorous region in the body of a mammal, wherein the method comprises the steps of administering, to a patient, a formulation comprising processed Morinda Citrifolia, wherein the processed Morinda citrifolia blocks formation of new blood vessels within a tumorous region by inhibiting tubule elongation and endothelial cell migration. Blocking the growth factors of said endothelial cells within said tumorous region inhibits tubule elongation and endothelial cell migration. In a preferred embodiment, the processed Morinda citrifolia comprises fruit juice and puree juice in various concentrations.

### BACKGROUND

1. Field of the Invention

The present invention relates to methods and formulations directed to the management of the cellular growth and the occurrence of natural formation or generation of blood vessels known as angiogenesis, and particularly to a unique method of reducing, inhibiting, and prohibiting angiogenesis within tumorous regions within the body, comprising the prophylactic administration of processed extracts or other forms of Morinda citrifolia.

2. Background of the Invention

Angiogenesis is a process known as the formation or generation of new blood vessels in the body and occurs primarily during wound healing stages and reproduction—associated neovascularisation or embryonic development. Angiogenesis is typically part of a healthy growth regime and is a normal and desirable occurrence considered essential for proper growth and homeostasis of the body and that provides the necessary blood vessels by which blood, nutrients, and other vital elements are delivered to the various parts of the body. However, under certain circumstances the natural effect of angiogenesis presents an undesirable threat by generating or forming new blood vessels that provides life support to abnormal growths or tumors and the harmful or diseased cells comprising these tumors. Pathological angiogenesis or abnormal proliferation of blood vessels has been implicated in over twenty diseases, including cancer, psoriasis, and age related macular degeneration. Stated differently, angiogenesis may be a significant contributor to the deadly spread, proliferation, or metastasizing of cancerous and other disease infected cells.

Cancer cells are often difficult to destroy. They can mutate, become resistant to drugs and spread unpredictably despite the best modern treatments. Moreover, current methods to attack cancer cells, such as chemotherapy and radiation, can also damage healthy tissues and often cause serious side effects. Anti-angiogenesis presents an exciting breakthrough in treating the above-identified diseases.

In normal tissue, new blood vessels are formed during tissue growth and repair, and the development of the fetus during pregnancy. In cancerous tissue, tumors cannot grow or spread (metastasize) without the development of new blood vessels as blood vessels serve to supply tissues with the oxygen and nutrients necessary for survival and growth.

The source of new blood vessels is endothelial cells. Endothelial cells form the walls of blood vessels and are capable of dividing and migrating towards the direction of the stimulus in the body. As such, endothelial cells constitute part of the building blocks of new blood vessels and are the primary focus of the present invention herein.

In short, to form or generate new blood vessels, an endothelial cell forming the wall of an existing blood vessel, particularly a small blood vessel such as a capillary, becomes activated, wherein it secretes enzymes known as MMP that degrade the extracellular matrix or the surrounding tissue. As the extracellular matrix is degraded, the endothelial cells invade the matrix and begin to divide. Eventually, the divided endothelial cells organize together into hollow tubes, thus creating new networks of blood vessels that make tissue growth and repair possible.

Normally, the endothelial cells are dormant but when activated, short bursts of blood vessel growth may occur in localized parts of tissues. New capillary growth is tightly controlled by a finely tuned balance between factors that activate endothelial cell growth and those that inhibit it. About 15 proteins are known to activate endothelial cell growth and movement, including angiogenin, epidermal growth factor, estrogen, fibroblast growth factors (acidic and basic), interleukin 8, prostaglandin E1 and E2, tumor necrosis factor, vascular endothelial growth factor (VEGF), and granulocyte colony-stimulating factor (G-CSF). Some of the known inhibitors of angiogenesis include angiostatin, platelet factor-4, thrombospondin-1, endostatin, angiopoietin2, prolactin, suramin, interferons, interleukin 1, interleukin 12, retinoic acid, and tissue inhibitor of metalloproteinase-1 and -2. (TIMP-1 and -2).

With regards to tumor growth and proliferation, at a critical point in its growth, the tumor sends out signals to the nearby endothelial cells to activate angiogenesis. Two endothelial growth factors, VEGF and basic fibroblast growth factor (bFGF), are expressed in larger amounts by many tumors and seem to be important in sustaining tumor growth.

As angiogenesis facilitates growth, it may also be said that angiogenesis is also related to metastasis. The metastasizing of disease-infected cells constitutes the spread or movement of diseased cells from the primary site to another area of the body. Other than certain white blood cells, this is something most normal cells cannot do, and it is the most deadly characteristic of diseases, such as cancer. During metastasis, tumor cells penetrate the fibrous boundaries that normally separate one tissue from another. The tumor can also infiltrate the walls of blood or lymph vessels and shed cancer cells into the circulation. In the blood, these tumor cells are carried to different parts of the body to become lodged in the next capillary bed where other tumors may be formed. Fewer than 1 in 10,000 cells shed from the primary tumor are thought to survive, but these are enough to spawn secondary tumors elsewhere in the body.

About 30 percent of new patients with solid tumors have detectable metastases. However, it is about half of the remaining patients will be cured by treating the tumor alone; the remainder will have undetectable metastases that will eventually develop into tumors. Tumor staging includes a measure of whether a malignancy has spread beyond the primary tumor. This is a major factor in determining a patient's prognosis.

It is generally true that tumors with higher densities of blood vessels are more likely to metastasize and are correlated with poorer clinical outcomes. Also, the shedding of cells from the primary tumor begins only after the tumor has a full network of blood vessels. In addition, both angiogenesis and metastasis require matrix metalloproteinases, enzymes that break down the surrounding tissue (the extracellular matrix), during blood vessel and tumor invasion.

The goal of early detection is to remove the primary tumor before significant growth or metastasis has occurred. Unfortunately, some tumors apparently metastasize before they are large enough to be found. It is well established that primary tumors send out inhibitors, which affect the growth of any secondary tumors and consequently the removal of the primary tumors and hence the inhibitors will allow the secondary tumors to growth. The spread of such micro-metastases may explain why many women die of breast cancer even after early detection of their primary tumors.

Recently, several experimental studies have focused on angiogenesis, its characteristic behavior, and its effects with regards to the proliferation and/or metastasizing of diseased cells. Indeed, angiogenesis within cancerous tumors, and particularly the possible discovery of anti-angiogenic solutions, has been among the priority area of study of the National Cancer Institute. The results of these studies have lead scientists to believe that abnormal growths or tumors, such as cancerous tumors, actually produce or generate new blood vessels, thus contributing to their growth factor, which growth factor permits such tumors to proliferate and metastasize, as well as to parlay non-cancerous tissues.

### SUMMARY AND OBJECTS OF THE INVENTION

The present invention provides a prophylactic regimen to inhibit, and in some instances stop, cancerous tumors before they parlay the non-cancerous tissues by blocking their ability to generate and form new blood vessels. The present invention features a method for preventing angiogenesis or the production of new blood vessels in disease infected cells and/or tumors, and particularly cancerous tumors, of mammals during the initial stages of tumor growth. The present invention further features a method for inhibiting further growth of the disease infected cells by limiting the number of blood vessels capable of providing nutrients and other life support to the tumorous region.

Morinda citrifolia is believed to have broad therapeutic effects including anticancer and growth prohibiting activity. Experiments conducted by the inventors indicates that Morinda citrifolia, as processed and administered according to the description herein, possesses an anti-angiogenic solution, or rather possesses anti-angiogenic effects, when prophylactically administered and applied to the tumorous regions within the body. In other words, the present invention Morinda citrifolia effectively inhibits and prevents the generation and formation of additional blood vessels within tumors, thus inhibiting and preventing further growth of the tumor.

The present invention functions to completely inhibit angiogenesis in the tubule elongation and the endothelial cell migration phases of angiogenesis at various concentrations and over varied amounts of time.

Specifically, the present invention features a method of inducing an anti-angiogenesis effect within a tumorous region in the body of a mammal, wherein the method comprises the steps of administering, to a patient, a formulation comprising processed Morinda Citrifolia, wherein the processed Morinda citrifolia blocks formation of new blood vessels within a tumorous region by inhibiting tubule elongation and endothelial cell migration. Tubule elongation and endothelial cell migration is inhibited by blocking the growth factors of said endothelial cells within said tumorous region. In a preferred embodiment, the processed Morinda citrifolia comprises fruit juice and puree juice in various concentrations.

The present invention further features an angiogenic inhibitor comprising a formulation comprised of processed Morinda citrifolia. The formulation, and particularly the Morinda citrifolia, effectuates the inhibition of tubule elongation and endothelial cell migration within a tumorous region by blocking the growth factors of the endothelial cells existing within the tumorous region.

### BRIEF DESCRIPTION OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 illustrates a bar graph showing tubule lengths of newly formed blood vessels, and particularly the difference in length of those tubules treated with Morinda citrifolia fruit juice at concentrations between 0.1 and 10 percent against other known inhibitors and untreated cells; and

FIG. 2 illustrates a bar graph showing tubule lengths of newly formed blood vessels, and particularly the difference in length of those tubules treated with Morinda citrifolia puree juice at concentrations between 0.1 and 10 percent against other known inhibitors and untreated cells.

### DETAILED DESCRIPTION OF THE PREFERRED EMBODIMENTS

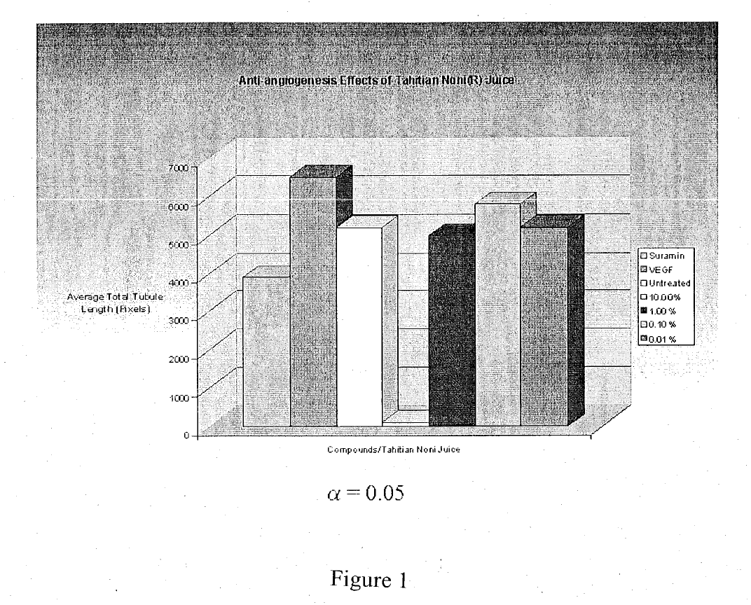

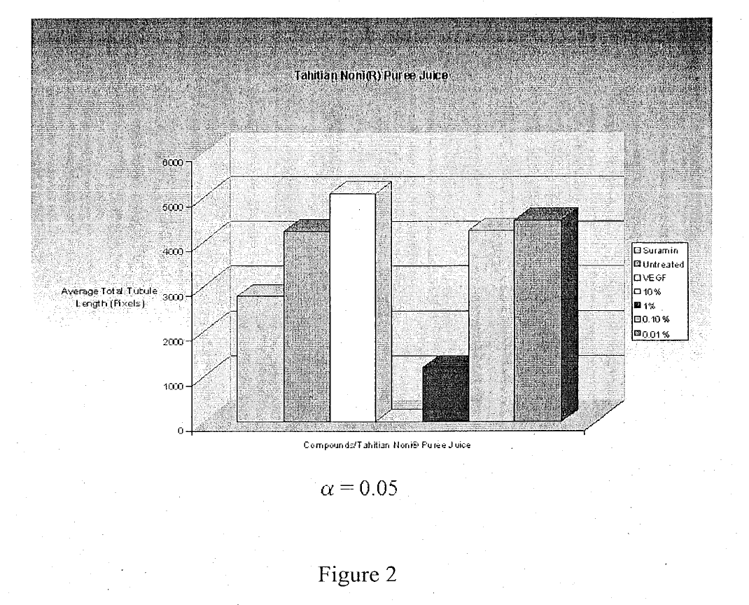

It will be readily understood that the components of the present invention, as generally described herein and illustrated in FIGS. 1 and 2, could be arranged and designed in a wide variety of different configurations. Thus, the following more detailed description of the embodiments of the methods and formulations of the present invention is not intended to limit the scope of the invention, as claimed, but is merely representative of the presently preferred embodiments of the invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

To more clearly point out and explain the present invention, the following more detailed description is divided into two sections, the first dealing with a general description of Morinda citrifolia, including the particular manufacturing processes and products produced from these processes; and the second dealing with the particular formulations comprising the Morinda citrifolia as processed and manufactured according to the methods described herein, and methods of application or administration of such formulations for the specific purpose of providing anti-angiogenic effects within tumorous regions in the body.

### General Discussion of Processing Morinda Citrifolia

The Indian Mulberry plant, known scientifically as Morinda citrifolia L., is a shrub, or small or medium sized tree growing three to ten meters in height. The Indian Mulberry plant is native to Southeast Asia and has spread in early times to a vast area from India to eastern Polynesia. Typically, the plant grows in tropical coastal regions around the world. The plant grows randomly in the wild, and it has thus been cultivated in many plantations and small individual growing plots.

The Indian Mulberry plant has somewhat rounded branches and evergreen, opposite (or spuriously alternate), dark, glossy, wavy, prominently-veined leaves. The leaves of the Indian mulberry plant are oppositely arranged with an elliptic to ovate form and pointed ends, and range from ten to thirty centimeters in length and five to 15 centimeters in width. The flowers are contained in a fleshy, globose, head-like cluster and are small, 3 to 5 lobed, tubular, fragrant, and about 1.25 cm long. The fruits are large, fleshy, and ovoid. The flowers mature and develop into compound fruits composed of many small drupes fused into an ovoid, ellipsoid or roundish, lumpy body, five to ten centimeters long, five to seven centimeters thick, with waxy, white or greenish-white or yellowish, semi-translucent skin. At maturity, they are edible, but have an unpleasant odor and a unique taste that many find very unpalatable. The fruit contains “eyes” on its surface, similar to a potato. The fruit is juicy, bitter, dull-yellow or yellowish-white, and contains numerous red-brown, hard, oblong-triangular, winged, 2-celled stones, each containing about four seeds.

When fully ripe, the fruit has a pronounced odor like rancid cheese. Although the fruit has been eaten by several nationalities as food, the most common use of the Indian mulberry plant was as a red and yellow dye source. Recently, there has been an interest in the nutritional and health benefits of the Indian mulberry plant.

Because the Morinda citrifolia fruit is for all practical purposes inedible, the fruit must be processed in order to make it palatable for human consumption and edible for inclusion into various food products and dietary supplements used to benefit the body as described and set forth herein. Processed Morinda citrifolia juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other processes include freeze drying the fruit and juice. The fruit and juice can be reconstituted during production of the final juice product. Still other processes include air drying the fruit and juices, prior to being masticated.

In a currently preferred process of producing Morinda citrifolia juice, the fruit is either hand picked or picked by mechanical equipment. The fruit can be harvested when it is at least one inch (2-3 cm) and up to twelve inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from zero to fourteen days, with most fruit being held from two to three days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and firmness. Spoiled and hard green fruit is separated from the acceptable fruit.

The ripened and aged fruit is preferably placed in plastic lined containers for further processing and transport. The containers of aged fruit can be held from zero to thirty days. Most fruit containers are held for seven to fourteen days before processing. Optionally, the containers can be stored under refrigerated conditions prior to further processing. Once ripe, the fruit is unpacked from the storage containers and is processed through a manual or mechanical separator, which separates the seeds and peel from the juice and pulp. The juice/pulp concentration is commonly referred to as a puree. Additionally, the juice can be filtered from the pulp if necessary.

Once separated, the pulp/juice combination (or puree) can be packaged into containers for storage and transport. The containers can be stored in refrigerated, frozen, or room temperature conditions. The homogenous blend of Morinda citrifolia juice and pulp puree can then be mixed with other ingredients to create various dietary supplements and other food products. These other ingredients may consist of, but are not limited to water, fruit juice concentrates, flavorings, sweeteners, nutritional ingredients, botanicals, and colorings. The finished juice product is preferably heated and pasteurized at a minimum temperature of 181° F. (83° C.) or higher up to 212° F. (100° C.).

Alternatively, the juice can be immediately processed into a finished juice product. The juice is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp are further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, reverse osmosis filtration., and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

Drying may further process the wet pulp. The methods of drying consist of but are not limited to freeze drying, drum drying, tray drying, sun drying, and spray drying. The dried Morinda citrifolia pulp preferably has a moisture content in the range from 0.1 to 15 percent by weight and more preferably from 5 to 10 percent by weight. The dried pulp preferably has a fiber content in the range from 0.1 to 30 percent by weight, and more preferably from 5 to 15 percent by weight.

The high fiber product typically includes, but is not limited to, wet or dry Morinda citrifolia pulp, supplemental fiber ingredients, water, sweeteners, flavoring agents, coloring agents, and nutritional ingredients. The supplemental fiber ingredients can include, but are not limited to plant based fiber products, either commercially available or developed privately. Examples of some typical fiber products are guar gum, gum arabic, soy bean fiber, oat fiber, pea fiber, fig fiber, citrus pulp sacs, hydroxymethylcellulose, cellulose, seaweed, food grade lumber or wood pulp, hemicellulose, etc. Other supplemental fiber ingredients may be derived from grains or grain products. The concentrations of these other fiber raw materials typically range from 0 up to 30 percent, by weight, and more preferably from 10 to 30 percent by weight.

Typical sweeteners include, but are not limited to, natural sugars derived from corn, sugar beet, sugar cane, potato, tapioca, or other starch-containing sources that can be chemically or enzymatically converted to crystalline chunks, powders, and/or syrups. Also sweeteners can consist of artificial or high intensity sweeteners, some of which are aspartame, sucralose, stevia, saccharin, etc. The concentration of sweeteners is preferably between from 0 to 50 percent by weight, of the formula, and more preferably between about 1 and 5 percent by weight.

Typical flavorings can include, but are not limited to, artificial and/or natural flavorings or ingredients that contribute to palatability. The concentration of flavorings is preferably from 0 up to 15 percent by weight, of the formula. Colors preferably include, but are not limited to, food grade artificial or natural coloring agents having a concentration ranging from 0 up to 10 percent by weight, of the formula.

Typical nutritional ingredients consist of but are not limited to vitamins, minerals, trace elements, herbs, botanical extracts, bioactive chemicals and compounds at concentrations from 0 up to 10 percent by weight. Examples of vitamins one can add to the fiber composition include, but are not limited to, vitamins A, B1 through B12, C, D, E, Folic Acid, Pantothenic Acid, Biotin, etc. Examples of minerals and trace elements one can add to the fiber composition include, but are not limited to, calcium, chromium, copper, cobalt, boron, magnesium, iron, selenium, manganese, molybdenum, potassium, iodine, zinc, phosphorus, etc. Herbs and botanical extracts include, but are not limited to, alfalfa grass, bee pollen, chlorella powder, Dong Quai powder, Echinacea root, Gingko Biloba extract, Horsetail herb, Indian mulberry, Shitake mushroom, spirulina seaweed, grape seed extract, etc. Typical bioactive chemicals can include, but are not limited to, caffeine, ephedrine, L-carnitine, creatine, lycopene, etc.

The juice and pulp can be dried using a variety of methods. The juice and pulp mixture can be pasteurized or enzymatically treated prior to drying. The enzymatic process begins with heating the product to a temperature between 75° F. and 135° F. It is then treated with either a single enzyme or a combination of enzymes. These enzymes include, but are not limited to, amylase, lipase, protease, cellulase, bromelin, etc. The juice and pulp can also be dried with other ingredients, such as those described above in connection with the high fiber product. The typical nutritional profile of the dried juice and pulp is 1 to 20 percent moisture, 0.1 to 15 percent protein, 0.1 to 20 percent fiber, and the vitamin and mineral content.

The filtered juice and the water from washing the wet pulp are preferably mixed together. The filtered juice is preferably vacuum evaporated to a brix of 40 to 70 and a moisture of 0.1 to 80 percent, more preferably from 25 to 75 percent. The resulting concentrated Morinda citrifolia juice may or may not be pasteurized. The juice would not be pasteurized in circumstances where the sugar content or water activity was sufficiently low enough to prevent microbial growth. It is packaged for storage, transport and/or further processing.

In addition to the processing methods described above, other methods of processing fruit into an oil product, a fiber product, and a juice product are contemplated and may be employed. Several embodiments of formulations of processed juice, oil, and fiber may be used. Some embodiments of the present invention encompass a method of treating various diseases and ailments that comprises administering to a mammal an effective amount of processed Morinda citrifolia.

The Morinda citrifolia plant is rich in natural ingredients. Those ingredients that have been discovered include: from the leaves: alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; from the flowers: acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; from the fruit: acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; from the roots: anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; from the root bark: alizarin, chlororubin, glycosides (pentose, hexose), morindadiol, morindanigrine, morindine, morindone, resinous matter, rubiadin monomethyl ether, and soranjidiol; from the wood: anthragallol-2,3-dimethylether; from the tissue culture: damnacanthal, lucidin, lucidin-3-primeveroside, and morindone-6beta-primeveroside; from the plant: alizarin, alizarin-alpha-methyl ether, anthraquinones, asperuloside, hexanoic acid, morindadiol, morindone, morindogenin, octanoic acid, and ursolic acid.

As mentioned, recently, many health benefits have been discovered stemming from the use of products containing Morinda citrifolia. The benefit of Morinda citrifolia is found in its ability to isolate and produce Xeronine, a relatively small alkaloid physiologically active within the body. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase that releases Xeronine from Proxeronine. A paper entitled, “The Pharmacologically Active Ingredient of Morinda citrifolia” by R. M. Heinicke of the University of Hawaii, indicates that Morinda citrifolia is “the best raw material to use for the isolation of Xeronine,” because of the building blocks of Proxeronine and Proxeronase. These building blocks aid in the isolation and production of Xeronine within the body. The function of the essential nutrient Xeronine is fourfold.

First, Xeronine serves to activate dormant enzymes found in the small intestines. These enzymes are critical to efficient digestion, calm nerves, and overall physical and emotional energy.

Second, Xeronine protects and keeps the shape and suppleness of protein molecules so that they may be able to pass through the cell walls and be used to form healthy tissue. Without these nutrients going into the cell, the cell can not perform its job efficiently. Without Proxeronine to produce Xeronine our cells, and subsequently the body, suffer.

Third, Xeronine assists in enlarging the membrane pores of the cells. This enlargement allows for larger chains of peptides (amino acids or proteins) to be admitted into the cell. If these chains are not used they become waste.

Fourth, Xeronine, which is made from Proxeronine, assists in enlarging the pores to allow better absorption of nutrients.

Each tissue has cells that contain proteins that have receptor sites for the absorption of Xeronine. Certain of these proteins are the inert forms of enzymes that require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

Because of its many benefits, Morinda citrifolia has been known to provide a number of anecdotal effects in individuals having cancer, arthritis, headaches, indigestion, malignancies, broken bones, high blood pressure, diabetes, pain, infection, asthma, toothache, blemishes, immune system failure, and others.

### Present Invention Anti-Angiogenesis Formulations and Methods of Administering the Same

In one exemplary embodiment, which is not meant to be limiting in any way, the beneficial Morinda citrifolia is processed into several products and forms, including Tahitian Noni® juice as manufactured by Morinda, Incorporated of Orem, Utah.

Preferably, to practice the invention, Morinda citrifolia is administered to a patient possessing an abnormal growth, such as a disease infected tumor, in an amount sufficient to prevent further growth of the tumor by specifically preventing the generation and formation of blood vessels within the tumorous region. Administering Morinda citrifolia to the patient prevents or stifles angiogenesis, wherein the tumor is prohibited from parlaying the non-cancerous tissue and metastasizing to further areas of the body.

In an exemplary embodiment, the preferred dosage is at least one ounce of Morinda citrifolia liquid administered twice daily. Greater doses do not create adverse or negative side effects, but have been found to even be quite beneficial to the patient in regards to their overall health and well being. For example, in one embodiment, up to one liter was administered daily with a significant prophylactic effect and no adverse side effects. Some anecdotal evidence also seems to indicate that remedial benefits may also be experienced.

The following formulations represent several exemplary formulations contemplated by the present invention.

| | |
|---|---|
| Ingredients | Percent by Weight |

Formulation One

| | |
|---|---|
| Morinda citrifolia Fruit Juice | 100% |

Formulation Two

| | |
|---|---|
| Morinda citrifolia Puree Fruit Juice | 100% |

Formulation Three

| | |
|---|---|
| Morinda citrifolia Fruit Juice | 15-99.9% |
| Water | 0.1-85% |

Formulation Four

| | |
|---|---|
| Morinda citrifolia Fruit Juice | 15-99.9% |
| Other fruit juices | 0.1-85% |

Formulation Five

| | |
|---|---|
| Morinda citrifolia Fruit Juice | 50-90% |
| Water | 0.1-50% |
| Other fruit juices | 0.1-30% |

Formulation Six

| | |
|---|---|
| Morinda citrifolia Puree Fruit Juice | 85-99.9% |
| Water | 0.1-15% |

In one preferred method, a person diagnosed with an abnormal growth as described above is administered at least one (1) ounce of Formulation One or Formulation Two in the morning on an empty stomach, and at least one (1) ounce at night on an empty stomach, just prior to retiring to bed.

In an alternative embodiment, a person experiencing angiogenesis within an abnormal growth is administered at least one ounce of Formulations Three, Four, Five, or Six twice a day until the growth is abated.

Through detailed clinical experiments, it has been found that Morinda citrifolia is capable of inhibiting and preventing angiogenesis, or the formation of blood vessels within the body. The following examples illustrate the results obtained from these experiments and are for illustrative purposes only. These examples are not meant to be limiting in any way as one ordinarily skilled in the art will recognize other various parameters and control groups that may be used to carry out the intended function of the present invention as intended herein.

### EXAMPLE ONE

As stated above, Morinda citrifolia possesses an anti-angiogenic effect. Specifically, Morinda citrifolia is capable of inducing anti-angiogenesis in abnormal growth regions within the body of a mammal, and particularly an anti-angiogenic effect at the initiation stage of tumor and other disease infected cell regions by preventing the further formation or generation of additional blood vessels through which the tumor or growth may obtain significant and vital life support from nutrients and other supplements found in the blood.

In this particular example, carefully processed Morinda citrifolia fruit juice was used as the active mechanism for inducing anti-angiogenic effects within endothelial cells contained within a controlled environment.

Cultures were examined for their morphology or signs of growth. On day 1, small islands of endothelial cells were visible in the cellular matrix. The culture was incubated at 37 degrees Celsius with 5% CO₂ humidified AT. At the end of day 1, the medium was changed and the culture further monitored.

On days 4, 7, and 9, the cultures were carefully examined for cell morphology and signs of growth. The media was aspirated and the test compounds of Morinda citrifolia added in the desired concentrations. The concentrations of Morinda citrifolia ranged between 0.1 and 10 percent by weight and consisted of Morinda citrifolia fruit juice. After the Morinda citrifolia test compounds were added, the cultures were incubated.

On days 10, 12, and 14, the cultures were carefully examined for tubule or blood vessel development. The mediums were aspirated, fixed, and stained with one or more endothelial markers, such as CD31 (PECAM-1) or von Willebrand factor.

The results of these experiments are as follows. Morinda citrifolia fruit juice completely inhibited tubule formation in the process of angiogenesis at a concentration of 10 percent by weight, and significantly inhibited tubule formation in the process of angiogenesis at concentrations between about 0.1 and 10 percent by weight of the total formulation. Indeed, tubule lengths were significantly smaller than the tubules formed within the control, non-treated culture (see FIG. 1).

These results suggest that Morinda citrifolia, as processed and produced according to the description provided herein, inhibits the elongation and migration phases of the angiogenic process with respect to the specified concentrations.

### EXAMPLE TWO

As stated above, Morinda citrifolia possesses an anti-angiogenic effect. Specifically, Morinda citrifolia is capable of inducing anti-angiogenesis in abnormal growth regions within the body of a mammal, and particularly an anti-angiogenic effect at the initiation stage of tumor and other disease infected cell regions by preventing the further formation or generation of additional blood vessels through which the tumor or growth may obtain significant and vital life support from nutrients and other supplements found in the blood.

In this particular example, carefully processed Morinda citrifolia puree juice was used as the mechanism for inducing anti-angiogenic effects within endothelial cells contained within a controlled environment.

Cultures were examined for their morphology or signs of growth. On day 1, small islands of endothelial cells were visible in the cellular matrix. The culture was incubated at 37 degrees Celsius with 5% CO₂ humidified AT. At the end of day 1, the medium was changed and the culture further monitored.

On days 4, 7, and 9, the cultures were carefully examined for cell morphology and signs of growth. The media was aspirated and the test compounds of Morinda citrifolia added in the desired concentrations. The concentrations of Morinda citrifolia ranged between 0.1 and 10 percent by weight and consisted of Morinda citrifolia puree juice. After the Morinda citrifolia test compounds were added, the cultures were incubated.

On days 10, 12, and 14, the cultures were carefully examined for tubule or blood vessel development. The mediums were aspirated, fixed, and stained with one or more endothelial markers, such as CD31 (PECAM-1) or von Willebrand factor.

The results of these experiments are as follows. Morinda citrifolia puree juice completely inhibited tubule formation in the process of angiogenesis at a concentration of 10 percent by weight, and significantly inhibited tubule formation in the process of angiogenesis at concentrations of between about 1 and 10 percent by weight of the total formulation. Indeed, tubule lengths were significantly smaller than the tubules formed within the control, non-treated culture (see FIG. 2).

These results suggest that Morinda citrifolia puree juice, as processed and produced according to the description provided herein, inhibits the elongation and migration phases of the angiogenic process with respect to the specified concentrations.

The present invention may be embodied in other specific forms without departing from its spirit of essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A method of inducing an anti-angiogenesis effect within a tumorous region in the body of a mammal, said method comprising the steps of:
administering, to a patient, a formulation comprising processed Morinda Citrifolia, wherein said Morinda citrifolia blocks formation of new blood vessels within a tumorous region by inhibiting tubule elongation and endothelial cell migration by blocking the growth factors of said endothelial cells within said tumorous region.

2. The method of claim 1, wherein said Morinda Citrifolia is administered in dosages comprising at least one ounce of said processed Morinda Citrifolia twice daily on an empty stomach for a period of at least two months.

3. The method of claim 1, wherein said processed Morinda Citrifolia is Morinda Citrifolia juice.

4. The method of claim 1, wherein said processed Morinda Citrifolia is Morinda Citrifolia puree juice.

5. The method of claim 1, wherein said Morinda Citrifolia is in powder form.

6. The method of claim 1, wherein said Morinda Citrifolia is in solid form.

7. The method of claim 1, wherein said formulation comprises said Morinda Citrifolia in an amount between about 0.1 and 5 percent by weight, said formulation being administered for a period of at least seven days.

8. The method of claim 1, wherein said formulation comprises said Morinda Citrifolia in an amount between about 5 and 10 percent by weight, said formulation being administered for a period of at least seven days.

9. The method of claim 1, wherein said formulation comprises said Morinda Citrifolia in an amount between about 10 and 15 percent by weight, said formulation being administered for a period of at least seven days.

10. The method of claim 1, wherein said formulation comprises said Morinda Citrifolia in an amount between about 15 and 20 percent by weight, said formulation being administered for a period of at least seven days.

11. The method of claim 1, wherein said formulation comprises said Morinda Citrifolia in an amount between about 20 and 25 percent by weight, said formulation being administered for a period of at least seven days.

12. The method of claim 1, wherein said formulation comprises said Morinda Citrifolia in an amount between about 25 and 30 percent by weight, said formulation being administered for a period of at least seven days.

13. The method of claim 1, wherein said formulation comprises said Morinda Citrifolia in an amount between about 30 and 35 percent by weight, said formulation being administered for a period of at least seven days.

14. The method of claim 1, wherein said formulation comprises said Morinda Citrifolia in an amount between about 35 and 40 percent by weight, said formulation being administered for a period of at least seven days.

15. The method of claim 1, wherein said formulation comprises said Morinda Citrifolia in an amount between about 40 and 45 percent by weight, said formulation being administered for a period of at least seven days.

16. The method of claim 1, wherein said formulation comprises said Morinda Citrifolia in an amount between about 45 and 50 percent by weight, said formulation being administered for a period of at least seven days.

17. The method of claim 1, wherein said formulation comprises said Morinda Citrifolia in an amount between about 50 and 55 percent by weight, said formulation being administered for a period of at least seven days.

18. The method of claim 1, wherein said formulation comprises said Morinda Citrifolia in an amount between about 55 and 60 percent by weight, said formulation being administered for a period of at least seven days.

19. The method of claim 1, wherein said formulation comprises said Morinda Citrifolia in an amount between about 60 and 65 percent by weight, said formulation being administered for a period of at least seven days.

20. The method of claim 1, wherein said formulation comprises said Morinda Citrifolia in an amount between about 65 and 70 percent by weight, said formulation being administered for a period of at least seven days.

21. The method of claim 1, wherein said formulation comprises said Morinda Citrifolia in an amount between about 70 and 75 percent by weight, said formulation being administered for a period of at least seven days.

22. The method of claim 1, wherein said formulation comprises said Morinda Citrifolia in an amount between about 75 and 80 percent by weight, said formulation being administered for a period of at least seven days.

23. An angiogenic inhibitor comprising:
a formulation comprising processed Morinda citrifolia, said Morinda citrifolia effectuating the inhibition of tubule elongation and endothelial cell migration within a tumorous region by blocking specific growth factors of said endothelial cells.

24. The angiogenic inhibitor of claim 23, wherein said formulation comprises processed Morinda citrifolia fruit juice.

25. The angiogenic inhibitor of claim 23, wherein said formulation comprises processed Morinda citrifolia puree juice.

26. The angiogenic inhibitor of claim 23, wherein said processed Morinda citrifolia is present in an amount between about 0.1 and 100 percent by weight.

27. The angiogenic inhibitor of claim 23, wherein said formulation further comprises water present in an amount between about 0.1 and 85 percent by weight.

28. A method for inhibiting and preventing angiogenesis within a tumorous region in the body of a mammal, said method comprising the steps of:
orally administering at least one ounce of a food product comprising processed Morinda citrifolia on an empty stomach in the morning, and
orally administering at least one ounce of said food product prior to sleeping at night, said Morinda citrifolia effectuating the inhibition of tubule elongation and endothelial cell migration within a tumorous region by blocking specific growth factors of said endothelial cells.

29. The method of claim 28, wherein said food product comprises:
processed Morinda citrifolia fruit juice present in an amount by weight of about 100 percent.

30. The method of claim 28, wherein said food product comprises:
processed Morinda citrifolia fruit juice present in an amount by weight between about 85-99.99 percent; and
water present in an amount by weight between about 0.1-15 percent.

31. The method of claim 28, wherein said food product comprises:
processed Morinda citrifolia fruit juice present in an amount by weight between about 85-99.99 percent; and
other fruit juices present in an amount by weight between about 0.1-15 percent.

32. The method of claim 28, wherein said food product comprises:
processed Morinda citrifolia fruit juice present in an amount by weight between about 50-90 percent;
water present in an amount by weight between about 0.1-50 percent; and
other fruit juices present in an amount between about 0.1-30 percent.

33. The method of claim 28, wherein said food product comprises:
processed Morinda citrifolia puree juice present in an amount by weight of about 100 percent.

34. The method of claim 28, wherein said food product comprises:
processed Morinda citrifolia puree juice present in an amount by weight between about 85-99.99 percent; and
water present in an amount by weight between about 0.1-15 percent.

35. The method of claim 28, wherein said food product is consumed by orally administering at least one ounce of said food product, twice daily until angiogenesis within said tumorous region is abated.
